(19) United States
(12) Patent Application Publication
(10) Pub. No.: US 2005/0152967 A1
(43) Pub. Date: Jul. 14, 2005

(54) Dynamic variable release

(75) Inventors: Mark Tengler, Colleyville, TX (US); Darlene Ryan, Fort Worth, TX (US)

Correspondence Address: CHALKER FLORES, LLP, 12700 PARK CENTRAL, STE. 455, DALLAS, TX, 75251, US

(73) Assignee: PFab, LP, Grand Prairie, TX (US)

(21) Appl. No.: 11/010,944

(22) Filed: Dec. 13, 2004

Related U.S. Application Data
(63) Continuation-in-part of application No. 10/764,177, filed on Jan. 23, 2004.
Application No. 10/764,177 is a continuation of application No. 10/402,858, filed on Mar. 28, 2003, now abandoned.

(51) Int. Cl. A61K 9/48; A61K 9/20; A61K 9/26; A61K 31/137

(52) U.S. Cl. 424/451; 424/464; 514/649

(57) ABSTRACT

The present invention relates to novel mixed release pharmaceutical formulations that include a expectorant available for immediate release and a decongestant for extended release that provide for the symptomatic relief of cough associated with respiratory tract conditions such as the common cold, bronchial asthma, acute and chronic bronchitis.

### FIELD OF INVENTION

The invention relates to novel mixed release pharmaceutical formulations having an expectorant for immediate release and a decongestant for mixed release, wherein the release profiles of the ingredients are controlled to maximize the effectiveness of their pharmacological action.

### BACKGROUND OF THE INVENTION

Without limiting the scope of the invention, its background is described in connection with immediate and extended release formulations and combination drug therapy, as an example. Heretofore, in this field, medications have been formulated so that they may be administered in a reduced number of daily doses. These doses must also provide drug that is released uniformly over a desired, extended period of time. Sustained or extended release pharmaceutical formulations provide a significant advantage over immediate release formulations to both clinicians and their patients because patients require fewer daily doses than their immediate release counterparts. In some cases, extended release formulation may improve therapeutic efficiency due to more consistent drug serum levels.

Various techniques have been developed to provide pharmaceutical preparations that include, e.g., a drug-containing particle with a coating layer and a pharmaceutical preparation in a continuous matrix with a drug dispersed therein, such as embedded into a rigid lattice of a resin. To achieve extended release, some pharmaceutical preparations include generally, a partially or completely insoluble matrix that in aqueous body fluids releases the drug. Alternatively, pharmaceutical preparations made of particles may be coated to provide extended release. It is believed that the release of the drug from such pharmaceutical preparations is driven by the gradient of the drug concentration resulting from penetration of water by diffusion into the formulation. The rate of the release decreases due to a decrease in the concentration gradient and the increase in the distance of diffusion. A sustained release formulation is also believed to help reduce side effects caused by a drug because they deliver the drug in slow, incremental amounts versus the cyclic high and low concentrations of immediate release formulations. By providing more consistent drug levels it is argued that the patient is better able to process the drug to avoid undesirable side-effects. Sustained release formulations for the sequential or timed release of medicaments are generally known in the art. Such formulations often contain drug particles mixed with or covered by a polymer material, or blend of materials, which is resistant to degradation or disintegration in the stomach and/or in the intestine for a selected period of time. Release of the drug may occur by leeching, erosion, rupture, diffusion or similar actions depending upon the nature of the polymer material or polymer blend used.

To improve the release profile of certain sustained release dosage forms, some formulations include tablets and capsules that include a combination of an immediate release formulation and a sustained release formulation. Although the inclusion of tablets and capsules improves control over the dosing of drug levels in the blood stream in some formulations, the extended therapeutic effect may not be improved or desired.

Furthermore, every active has different solubility properties and pH dependencies that affect, e.g., its dissolution rate, and hence its bioavailability. Bioavailability may also be affected by a number of factors such as the amounts and types of additives used, its granulation and compression, surface area, mechanical shearing (e.g., by the stomach), pH, solubility of the active agent in water, the presence of food, etc. Due to these numerous factors, the specific form of the drug, its excipients, coating, pH, dissolution profile alone, and in combination, affect the manner and formulation of actives to achieve the best bioavailability profile to achieve an optimum therapeutic effect.

U.S. Pat. Nos. 4,309,404 and 4,248,857 to DeNeale, et al., disclose slow release formulations formed of a core material containing the active drug, carboxypolymethylene, zinc oxide, stearic acid, and mannitol; a seal coating surrounding the core; and a sugar coating surrounding the seal coating. U.S. Pat. No. 4,309,405 to Guley, et al., discloses a sustained release tablet similar to that disclosed by DeNeale, et al., except that the core contains a drug, a mixture of a water-soluble polymer such as hydroxypropylmethylcellulose or hydroxypropylcellulose and a water-insoluble polymer (ethylcellulose alone or in admixture with carboxypolymethylene, hydroxypropylcellulose and the like). The DeNeale and Guley patents disclose that their compositions provide substantially zero order release of the core contained drug for about 12 hours following the first hour of administration. Thus, zero order release is only obtained after the initial surge of release of drug in the first hour.

U.S. Pat. No. 4,695,467 to Uemura, et al., relates to a sustained release tablet that includes easily disintegrable granules including: a drug, a disintegrating agent selected from the group consisting of starch derivatives, gums, cellulose derivatives and ion-exchange resins, and a water-soluble polymer selected from the group consisting of cellulose derivatives, synthetic water soluble polymers and polysaccharides. The surfaces of the granules are treated with a wax selected from the group consisting of plant or animal wax, hydrogenated oils and paraffin.

U.S. Pat. No. 6,372,252 to Blume, et al., relates to guaifenesin sustained release formulation and tablets that require a hydrophilic polymer and a water-insoluble polymer. The formulation is said to be capable of providing therapeutically effective bioavailability of guaifenesin for at least twelve hours after dosing in a human subject. The invention also relates to a modified release guaifenesin tablet that has two portions: the first portion comprises an immediate release formulation of guaifenesin and the second portion comprises a sustained release formulation of guaifenesin as described above. A two portion, or bi-layer, tablet has a maximum serum concentration equivalent to that of an immediate release guaifenesin tablet, and is capable of providing therapeutically effective bioavailability of guaifenesin for at least twelve hours after dosing in a human subject.

U.S. Pat. No. 6,462,094 to Dang, et al., relates to decongestant/expectorant compositions consisting essentially of phenylephrine tannate and guaifenesin that are effective when administered orally for the symptomatic relief of cough associated with respiratory tract conditions such as the common cold, bronchial asthma, acute and chronic bronchitis are disclosed.

### SUMMARY OF THE INVENTION

It has been found, however, that the present methods fail to provide an efficacious amount of an expectorant in an immediate release form and a decongestant that is provided as an extended release formulation that takes advantage of the pharmacological effect of the immediate release active to maximize the efficiency of the delivery and pharmacological action of the decongestant. Yet another problem is that certain drugs affect the release profile of a second drug that is being provided in a single dose. The present invention solves these problems in the art.

The present invention also addresses a growing concern for physicians as they write prescriptions for drugs: cost. While pharmacists continue to substitute generics in order to reduce cost to the patients or allow for greater insurance coverage, the effectiveness of dosing and effect has become paramount. The present invention increases the effectiveness of the individual components, thereby reducing the number of doses and increasing the therapeutic effectiveness. It may also be used to decrease dose sizes, thereby reducing costs. In one example of the advantages of the present invention, an expectorant (e.g., gauifenesin) is provided at lower doses and is made available immediately for absorption, followed by a lower dose of a decongestant (e.g., phenylephrine) which is release slowly over, e.g., about 90 minutes to about 8 hrs. This release profile makes the product more efficacious since the large amount of expectorant begins to break up mucus and the time released decongestant provides long acting decongestant activity.

One embodiment of the present invention is a capsule that includes an expectorant available for immediate release and a decongestant for extended release. The expectorant may be, e.g., gauifenesin that is compressed into a slug of between about 50, 200, 400, 500, 600 or more milligrams and packaged for release of over 90% of the active within about 90 minutes. The decongestant comprises a nasal decongestant, e.g., phenylephrine packaged as a sustained release bead, e.g., from between about 1.5 to 30 mg. The term immediate release is defined as release of over 90% within about 90 minutes. The decongestant for extended release provides between about 40 to 60% of the decongestant after 90 minutes, between about 50 to 70 percent at between 150 and 210 minutes and wherein between about 60 to 80 percent after 360 minutes.

In another embodiment, the present invention is a single pharmaceutical composition that includes an expectorant that is packed for immediate release; and a decongestant that is a nasal decongestant packed for extended release, wherein the expectorant provides productive coughs in the short-term and the decongestant provides long-acting decongestant activity.

In yet another embodiment, the present invention provides a time released phenylephrine that is formulated to provide maximum effective release over 2-8 hours using a combination of polymers and/or pharmaceutical glaze. It was found that when the phenylephrine were overcoated with immediate release gauifenesin the process was not only time consuming (since building up the bead with gauifenesin had adhesion problems), but also that overcoating of the gauifenesin on the phenylephrine slowed the release of the phenylephrine to an unacceptable level. Further attempts to increase adhesion by sustain releasing both actives also resulted in a poor release profile for gauifenesin. Nevertheless, overcoating the extended release active with an immediate release active may be used with these or other actives, depending on the actives selected and the desired efficacy. One embodiment of the present invention includes powder filling the gauifenesin and extended release phenylephrine into a capsule. The solution provided herein addresses the problems of dosing, effective pharmacological serum levels and cost. This process also reduces greatly the already taxed capacity on the bead room since up to about 96% of the active load would not need to go through the coating process.

In one embodiment, the present invention includes a pharmaceutical composition having an expectorant packaged for release of over 90% within about 90 minutes; and a decongestant packaged for extended release wherein between about 40 to 60% of the decongestant is available after 90 minutes, between about 50 to 70 percent is available at between 150 and 210 minutes and wherein between about 60 to 80 percent is available after 360 minutes. In another embodiment, the present invention is a method of providing a dual-release formulation that includes an expectorant packed for immediate release in a powder form, a nasal decongestant packed for extended release, e.g., phenylephrine, PVP, cellulose and a pharmaceutical glaze, and loading the expectorant and the decongestant into a capsule. Yet another embodiment is a pharmaceutical composition with an expectorant (e.g., guaifenesin) packaged for release of over 90% within about 90 minutes and a nasal decongestant (e.g., phenylephrine HCl) packed for extended release with PVP, microcrystalline cellulose and a pharmaceutical glaze, wherein between about 40 to 60% of the decongestant is available after 90 minutes, between about 50 to 70 percent is available at between about 150 and 210 minutes and wherein between about 60 to 80 percent is available after 360 minutes; and one or more inactive agents.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows one embodiment of the present invention for immediate release guaifenesin and delayed release phenylephrine HCl;

FIG. 2 is a graph that shows another embodiment of the present invention for immediate release guaifenesin and delayed release phenylephrine HCl; and

FIG. 3 is a graph that shows another embodiment of the present invention for immediate release guaifenesin and delayed release phenylephrine HCl.

### DETAILED DESCRIPTION OF THE INVENTION

While the making and using of various embodiments of the present invention are discussed in detail below, it should be appreciated that the present invention provides many applicable inventive concepts which can be embodied in a wide variety of specific contexts. The specific embodiments discussed herein are merely illustrative of specific ways to make and use the invention and do not delimit the scope of the invention.

The present invention is based on the recognition that patients and physicians are looking to simplify the number of doses that a patient takes, improving the efficacy of drug delivery and reducing costs. The effectiveness of dosing and effect has become paramount in order to reduce cost to the patients and allow for greater insurance coverage, while improving patient compliance.

Definitions

A number of definitions are provided herein to facilitate an understanding of the present invention. As used herein, the term “enveloped pharmaceutical” means a capsule, a suppository, a gel cap, a softgel, a lozenge, a sachet or even a fast dissolving wafer. As used herein the term “carrier” is used to describe a substance, whether biodegradable or not, that is physiologically acceptable for human or animal use and may be pharmacologically active or inactive.

The term “immediate release” as used herein is used to describe a release profile to effect delivery of an active as soon as possible, that is, as soon as practically made available to an animal, whether in active form, as a precursor and/or as a metabolite. Immediate release may also be defined functionally as the release of over 80 to 90 percent (%) of the active ingredient within about 60, 90, 100 or 120 minutes or less. Immediate release as used herein may also be defined as making the active ingredient available to the patient or subject regardless of uptake, as some actives may never be absorbed by the animal. Immediate release formulations of the active on a carrier, such as rolled or compressed beads, may be formulated such that the surface area is maximized on beads and the active is exposed immediately. The immediate release formulations may also include effervescing agents that cause the disintegration of the structure integrity of the active and carrier such that release of the active is maximized. Various immediate release dosage forms may be designed readily by one of skill in art to achieve drug delivery to the stomach and small intestine, depending upon the choice of compression, adhesive materials and/or beading.

The terms “extended release” and “delayed release” as used herein is used to define a release profile to effect delivery of an active over an extended period of time, defined herein as being between about 60 minutes and about 2, 4, 6 or even 8 hours. Extended release may also be defined functionally as the release of over 80 to 90 percent (%) of the active ingredient after about 60 minutes and about 2, 4, 6 or even 8 hours. Extended release as used herein may also be defined as making the active ingredient available to the patient or subject regardless of uptake, as some actives may never be absorbed by the animal. Various extended release dosage forms may be designed readily by one of skill in art as disclosed herein to achieve delivery to both the small and large intestines, to only the small intestine, or to only the large intestine, depending upon the choice of coating materials and/or coating thickness.

“Extended release” and “delayed release” formulations may be prepared and delivered so that release is accomplished at some generally predictable location in the lower intestinal tract more distal to that which would have been accomplished if there had been no delayed release alterations. A method for delay of release is, e.g., a coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers and compatible mixtures thereof may be used to provide the coating for the delayed or the extended release of active ingredients, and some of their properties, include, but are not limited to: shellac, also called purified lac, a refined product obtained from the resinous secretion of an insect. This coating dissolves in media of pH>7.

The present pharmaceutical composition may also be provided in a variety of dosage forms, e.g., solution, suspension, cream, ointment, lotion, capsule, caplet, softgel, gelcap, suppository, enema, elixir, syrup, emulsion, film, granule, gum, insert, jelly, foam, paste, pastille, pellet, spray, troche, lozenge, disk, magma, poultice, or wafer and the like.

For gelcap preparations, the pharmaceutical formulation may include oils, e.g.: (1) fixed oils, such as peanut oil, sesame oil, cottonseed oil, corn oil and olive oil; (2) fatty acids, such as oleic acid, stearic acid and isostearic acid; and fatty acid esters, such as ethyl oleate, isopropyl myristate, fatty acid glycerides and acetylated fatty acid glycerides; (3) alcohols, such as ethanol, isopropanol, hexadecyl alcohol, glycerol and propylene glycol; (4) glycerol ketals, such as 2,2-dimethyl-1,3-dioxolane-4-methanol; (5) ethers, such as poly(ethylene glycol) 450; (6) petroleum hydrocarbons, such as mineral oil and petrolatum; and (7) water, or with mixtures thereof; with or without the addition of a pharmaceutically suitable surfactant, suspending agent or emulsifying agent.

For oral, buccal, and sublingual administration, the pharmaceutical composition of the invention may be administered as either solutions or suspensions in the form of gelcaps, caplets, tablets, capsules or powders. For rectal administration, the compounds of the invention may be administered in the form of suppositories, ointments, enemas, tablets and creams for release of compound in the intestines, sigmoid flexure and/or rectum. For example, when making a suppository a beeswax/glycerol composition may be used to form a body meltable suppository for transrectal or transurethral delivery.

It is contemplated that the “immediate release” active may be formulated as, e.g., freeze dried, rotary dried or spray dried powders; amorphous or crystalline powders; granules, precipitates or particulates. The immediate release active may be either free-flowing or compressed. The pharmaceutical formulation may further include, e.g., water, aqueous solvents, non-protic solvents, protic solvents, hydrophilic solvents, hydrophobic solvents, polar solvents, non-polar solvent, emollients and/or combinations thereof. Other formulations may include, optionally, stabilizers, pH modifiers, surfactants, perfumes, astringents, cosmetic foundations, pigments, dyes, bioavailability modifiers and/or combinations thereof.

The immediate release actives of the present invention may be processed by agglomeration, air suspension chilling, air suspension drying, balling, coacervation, coating, comminution, compression, cryopelletization, encapsulation, extrusion, wet granulation, dry granulation, homogenization, inclusion complexation, lyophilization, melting, microencapsulation, mixing, molding, pan coating, solvent dehydration, sonication, spheronization, spray chilling, spray congealing, spray drying, or other processes known in the art. The extended release actives may be provided in the form of a minicapsule, a capsule, a tablet, an implant, a troche, a lozenge (minitablet), a temporary or permanent suspension, a pellet, a bead, a pill, a strip or a sachet.

The pharmaceutical composition and/or the solid carrier particles may be coated with one or more enteric coatings, seal coatings, film coatings, barrier coatings, compress coatings, fast disintegrating coatings, or enzyme degradable coatings. Multiple coatings may be applied for desired performance. For example, the pharmaceutical composition, e.g., phenylephrine may be mixed with one or more agents that delay release until the proper pH, gel formation and/or timed-release polymers and/or additives are provided. Further, some actives may be provided for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The compositions may be formulated for oral, nasal, buccal, ocular, urethral, transmucosal, vaginal, topical or rectal delivery, although oral delivery is used mostly.

For example, suitable mixed or extended release polymers for use with the present invention include but are not limited to synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll®, polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof.

The blend of polymers may form a hydrogel or matrix using a material such as a carbohydrate polymer or polysaccharide (e.g., hyaluronic acid) in the presence of an initiator such as mono-, di- or trivalent cations or anions in water, a radical, or a photoinitiator. The polymer blend may be intrinsically biodegradable, biocompatible, or of sufficiently low molecular weight to allow excretion. Some components of the polymer blend exhibit little to no ability to biologically degrade. Where there are two or more water-soluble polymer blocks joined by other groups, the joining groups may include biodegradable linkages, polymerizable linkages, or both.

Other polymer formulations for use with the present invention include scaffolds prepared with the polymer of the present invention and one or more bioactive compounds or active species so that the polymer or scaffold becomes a microcarrier for one or more active species. The active species may be incorporated into the polymer or polymer solution (e.g., scaffold) or may be attached to its surface using techniques readily apparent to those skilled in the art. In some instances, it may be preferred to incorporate or attach a precursor of the active agent, e.g., an inactive version of the species that can then be activated to the active species as needed and required. The active species may be a drug or other biologically active compound; thus, the scaffold may be a microcarrier for the delivery of drugs or other biologically active compounds when used in the body. Examples of biologically active compounds are proteins, peptides, polysaccharides, nucleic acids, oligonucleotides, natural and synthetic organic or inorganic molecules, and those biologic molecules used for therapeutic, prophylactic or diagnostic purposes. Drugs may include antibiotics, antivirals, chemotherapeutic agents, anti-angiogenic agents, hormones, anti-inflammatory agents, drugs having an effect on vascular flow or that are effective against one or more diseases, and combinations thereof.

When formulated as a capsule, the capsule can be a hard or soft gelatin capsule, a starch capsule, or a cellulosic capsule. Although not limited to capsules, such dosage forms may be further coated with, for example, a seal coating, an enteric coating, an extended release coating, or a targeted delayed release coating. For example, the capsule may include one or more actives in powder form. The term “powder” as used herein include, e.g., true powder, as well as truly crystalline materials, microgranulated, nanosprayed, nanoprecipitated, microprecipitated and/or granulated materials, agglomerates, adsorbates and the like. In addition, when these powders are coated, the coating contemplated is a rapid release coating. For immediate release of an active, suitable coatings (if any) will dissolve, disintegrate and/or become sufficiently porous to allow the full release and dissolution of the coated drug in a manner consistent with the administration of the same drug in a completely uncoated fashion. Certainly, the use of these “coated powders” should not alter the dissolution rates of the drug in the digestive tract by more than an hour and preferably by less than half an hour.

Dosage forms of the compositions of the present invention can also be formulated as enteric coated delayed release oral dosage forms, i.e., as an oral dosage form of a pharmaceutical composition as described herein that uses an enteric coating to effect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The coating may also contain a plasticizer and possibly other coating excipients such as colorants, talc, and/or magnesium stearate, which are well known in the art. Suitable plasticizers include: triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the lower intestinal tract is reached.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

Immediate release coating of solid carriers is commonly used to improve product elegance as well as for a moisture barrier, and taste and odor masking. Rapid breakdown of the film in gastric media is important, leading to effective disintegration and dissolution. Eudragit RD100 (Rohm) is an example of such a coating. It is a combination of a water insoluble cationic methacrylate copolymer with a water-soluble cellulose ether. In powder form, it is readily dispensable into an easily sprayable suspension that dries to leave a smooth film. Such films rapidly disintegrate in aqueous media at a rate that is independent of pH and film thickness.

Actives. Decongestants useful with the present invention (along with a salt form) are phenylephrine (bitartrate, tannate, HBr, HCl), phenylpropanolamine (HCl) and pseudoephedrine (HCl). Furthermore, a number of herbal and/or natural decongestants are known in the art, all of which may be used with the present invention.

Expectorants for use with the present invention include, e.g., guaifenesin, terpin hydrate, (glyceryl guaiacolate), potassium (iodide, citrate) and potassium guaicolsulfonate. Other expectorants, whether individual ingredients or combinations of ingredients may be used with the present invention. Furthermore, a number of herbal and/or natural expectorants are known in the art, all of which may be used with the present invention, e.g., Oregano Leaf Extract 25-500 mg (which may be a liquid extract), Red Clover 25-500 mg, Buckthorn Root 25-500 mg, or Fenugreek 25-500 mg, or mixtures thereof.

Examples of antihistamines for use with the present invention (e.g., in salt form) are chlorpheniramine (maleate), brompheniramine (maleate), dexchlorpheniramine (maleate), dexbrompheniramine (maleate), triprolidine (HCl), diphenhydramine (HCl), doxylamine (succinate), tripelennamine (HCl), cyproheptatine (HCl), bromodiphenhydramine (HCl), phenindamine (tartrate), pyrilamine (maleate, tannate) and azatadine (maleate). Antitussives that may be used with the present invention (with salt form) include: caramiphen (edisylate), dextromethorphan (HBr) and codeine (phosphate, sulfate). A number of herbal and/or natural antihistamines are known in the art, all of which may be used with the present invention.

Other actives may also be included with the present invention, e.g., non-steroidal anti-inflammatory drugs (NSAIDs) such as propionic acid derivatives; acetic acid derivatives; fenamic acid derivatives; biphenylcarboxylic acid derivatives; and oxicams. Examples of propionic acid derivatives include: ibuprofen, naproxen, ketoprofen, flurbiprofen, fenoprofen, suprofen, fenbufen, and fluprofen may be mentioned as preferred compounds. Acetic acid derivatives include: tolmetin sodium, zomepirac, sulindac and indomethacin. Fenamic acid derivatives include: mefenamic acid and meclofenamate sodium. Diflunisal and flufenisal are biphenylcarboxylic acid derivatives, while oxicams include piroxicam, sudoxicam and isoxicam. Other analgesics for use with the present invention include acetaminophen and phenacetin.

Those skilled in the art will appreciate that any of the foregoing compounds may be used in the form of their pharmaceutically acceptable salt forms, e.g.—carboxylic acids with potassium or sodium counter-ions, and the like. In one example of the present invention, an expectorant (e.g., Gauifenesin DC) is provided at lower doses and is made available immediately for absorption, followed by a lower dose of a decongestant (e.g., phenylephrine) which is release slowly over, e.g., about 1 to 8 hrs. This release profile makes the product more efficacious since the large amount of expectorant begins to break up mucus prior to the time the decongestant is released to provide long acting decongestant activity after mucus breakdown has begun. Generally, guaifenesin is present in amounts of about 10 to about 600 milligrams per capsule. Guaifenesin may be present in amounts of 100, 150, 200, 300, 400, 440, 500 or even 600 or more milligrams per capsule. In one example, guaifenesin is present in amounts of about 100 to about 200 milligrams per capsule, with half or less of that amount used in a pediatric form of the formulation.

In one example, 400 milligrams of gauifenesin are included as an active for immediate release. Guaifenesin is an expectorant that increases the output of phlegm (sputum) and bronchial secretions by reducing adhesiveness and surface tension. The increased flow of less viscous secretions promotes cilliary action and facilitates the removal of mucus. Hence, expectorants such as guaifenesin change a dry, unproductive cough to one that is more productive and less frequent. Guaifenesin, known chemically as 3(2-methoxyphenoxy)-1,2-propanediol, is a crystalline powder soluble in water and alcohol. It is indicated in the USP Drug information as an expectorant for the symptomatic relief of cough due to colds and minor upper respiratory infections.

Phenylephrine may be present in amounts of between about 15 and about 60 milligrams per capsule. Phenylephrine is generally in amounts of about 5 to about 30 milligrams per capsule, with half or less of that amount used in a pediatric form of the formulation. In one example of the present invention, phenylephrine is provided in the amount of about 15 mg for extended release. Phenylephrine hydrochloride is an orally effective nasal decongestant. Chemically it is (S)-3-hydroxy-α[(methylamino) methyl]benzenemethanol hydrochloride. Phenylepherine is a synthetic, optically active sympathomimetic amine that has one hydroxyl group on the benzene ring. The hydroxyl group is placed in the position meta to the aliphatic side chain. The meta position affords optimal activity and phenylepherine (neo-synephrine) replaced an older preparation, synephrine, in which the hydroxyl was in the para position. Phenylephrine hydrochloride is available in the form of the levorotatory isomer, a white, odorless, non-hygroscopic, crystalline compound possessing a bitter taste. Phenylephrine hydrochloride has a melting point of 140-145 degrees C and is freely soluble in water and alcohol. Decongestant compounds in the form of their free bases as well as their salts, e.g., hydrochloride, citrate, maleate, tannate, etc., are well known.

Excipients for use with the present invention are well known to those of skill in the art and include humectants such as glycerin and propylene glycol, preservatives such as sodium benzoate and paraben, sweeteners such as sodium saccharin, corn syrup and sorbitol solutions, menthol and various flavoring and coloring agents. The pharmaceutically active compounds and excipients for human use should be of N.F. or U.S.P. grade.

Sugar spheres may be used as inert cores in capsule and tablet formulations particularly multiparticulate sustained release formulations and are provided in amounts sufficient to accept the active ingredient for extended release, e.g., phenylephrine. Sugar spheres are generally of relatively uniform diameter and contain 62.5%-91.5% sucrose with the remainder being starch.

Pharmaceutical Glaze: (4.5 mg) Shellac is a natural occurring material, consisting of a complex mixture of constituents. The main component of shellac (˜95%) is a resin that upon mild basic hydrolysis gives a mixture of compounds of high plasticity. Shellac is used extensively in the pharmaceutical industry as a film coating agent for beads and tablets.

Substrate(s) for use with the present invention may be a powder or a multiparticulate, such as a granule, a pellet, a bead, a spherule, a beadlet, a microcapsule, a millisphere, a nanocapsule, a nanosphere, a microsphere, a platelet, a minitablet, a tablet or a capsule. A powder may be a finely divided (milled, micronized, nanosized, precipitated, sprayed) to form of an active ingredient or additive molecular aggregates or a compound aggregate of multiple components or a physical mixture of aggregates of an active ingredient and/or additives. Such substrates may be formed of various materials known in the art, such as, for example: sugars, such as lactose, sucrose or dextrose; polysaccharides, such as maltodextrin or dextrates; starches; cellulosics, such as microcrystalline cellulose or microcrystalline cellulose/sodium carboxymethyl cellulose; inorganics, such as dicalcium phosphate, hydroxyapitate, tricalcium phosphate, talc, or titania; and polyols, such as mannitol, xylitol, sorbitol or cyclodextrin.

It should be emphasized that a substrate need not be a solid material, although often it will be a solid. For example, the encapsulation coat on the substrate may act as a solid “shell” surrounding and encapsulating a liquid, semi-liquid, powder or other substrate material. Such substrates are also within the scope of the present invention, as it is ultimately the carrier, of which the substrate is a part, which must be a solid.

Excipients. Solid pharmaceutical compositions may include optionally one or more additives, sometimes referred to as excipients or additives. The excipients may be contained in an encapsulation coat in compositions, which include an encapsulation coat, or can be part of the solid carrier, such as coated to an encapsulation coat, or contained within the components forming the solid carrier. Alternatively, the excipients can be contained in the pharmaceutical composition but not part of the solid carrier itself.

Suitable excipients are those used commonly to facilitate the processes involving the preparation of the solid carrier, the encapsulation coating, or the pharmaceutical dosage form. These processes include agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, or other processes known in the art. The excipients may also be pre-coated or encapsulated, as are well known in the art.

The pharmaceutical compositions of the present invention may include optionally one or more solubilizers, i.e., additives to increase the solubility of the pharmaceutical active ingredient or other composition components in the solid carrier. It has been recognized by the present inventors that guaifenesin, in fact, acts as a solubilizer for phenylephrine, and is used as such in the examples provided herein. Other solubilizers are known in the art. Mixtures of solubilizers are also within the scope of the invention and are readily available from standard commercial sources.

The amount of solubilizer that may be included in compositions of the present invention is not particularly limited. Of course, when such compositions are administered to a patient, the amount of a given solubilizer is limited to a bioacceptable amount, which is readily determined by one of skill in the art. In some circumstances, it may be advantageous to include amounts of solubilizers far in excess of bioacceptable amounts, for example, to maximize the concentration of active ingredient, with excess solubilizer removed prior to providing the composition to a patient using conventional techniques, such as distillation or evaporation.

Other additives conventionally used in pharmaceutical compositions may be included, which are well known in the art. Such additives include, e.g.,: anti-adherents (anti-sticking agents, glidants, flow promoters, lubricants) such as talc, magnesium stearate, fumed silica), micronized silica, polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate.

Other additives include, binders (adhesives), i.e., agents that impart cohesive properties to powdered materials through particle-particle bonding, such as matrix binders (dry starch, dry sugars), film binders (polyvinylpyrrolidone (PVP), starch paste, celluloses, bentonite and sucrose), and chemical binders (polymeric cellulose derivatives, such as carboxy methyl cellulose, HPC and HPMC; sugar syrups; corn syrup; water soluble polysaccharides such as acacia, tragacanth, guar and alginates; gelatin; gelatin hydrolysate; agar; sucrose; dextrose; and non-cellulosic binders, such as PVP, PEG, vinyl pyrrolidone copolymers, pregelatinized starch, sorbitol, and glucose).

For certain actives it may be useful to provide buffering agents (or bufferants), where the acid is a pharmaceutically acceptable acid, such as hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, and where the base is a pharmaceutically acceptable base, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or a salt of a pharmaceutically acceptable cation and acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid.

In some formulations additives may also include: chelating agents (such as EDTA and EDTA salts); colorants or opaquants (such as titanium dioxide, food dyes, lakes, natural vegetable colorants, iron oxides, silicates, sulfates, magnesium hydroxide and aluminum hydroxide); coolants (e.g., trichloroethane, trichloroethylene, dichloromethane, fluorotrichloromethane); cryoprotectants (such as trehelose, phosphates, citric acid, tartaric acid, gelatin, dextran and mannitol); and diluents or fillers (such as lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose).

Yet other additives may include disintegrants or super disintegrants; hydrogen bonding agents, such as magnesium oxide; flavorants or desensitizers; ion-exchange resins, such as styrene/divinyl benzene copolymers, and quaternary ammonium compounds; plasticizers, such as polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate; and preservatives, such as ascorbic acid, boric acid, sorbic acid, benzoic acid, and salts thereof, parabens, phenols, benzyl alcohol, and quaternary ammonium compounds.

It should be appreciated that there is considerable overlap between the above-listed additives in common usage, since a given additive is often classified differently by different practitioners in the field, or is commonly used for any of several different functions. Thus, the above-listed additives should be taken as merely exemplary, and not limiting, of the types of additives that can be included in compositions of the present invention. The amounts of such additives may be readily determined by one skilled in the art, according to the particular properties desired.

The compositions of the present invention may be prepared by a variety of processes to apply an encapsulation coat onto a substrate or to form a substrate-free solid carrier such as a multiparticulate or a powder. The most commonly used coating and pelletization processes include: balling, spheronization, extrusion, spray congealing, spray drying, pan coating, fluidized bed coating, melt extrusion, crystallization, cryopelletization, nanoencapsulation, coacervation, spraying, precipitation, etc. One skilled in the art will recognize that appropriate additives may also be introduced to the composition or during the processes to facilitate the preparation of the solid carrier or the dosage forms, depending on the need of the individual process.

A coating process frequently involves spraying a coating solution onto a substrate. The coating solution can be a molten solution of the encapsulation coat composition free of a dispersing medium. The coating solution may also be prepared by solubilizing or suspending the composition of the encapsulation coat in an aqueous medium, an organic solvent, a supercritical fluid, or a mixture thereof. At the end of the coating process, the residual dispersing medium can be further removed to a desirable level using appropriate drying processes, such as vacuum evaporation, heating, freeze drying, etc.

A pelletization process typically involves preparing a molten solution of the composition of the solid carrier or a dispersion of the composition of the solid carrier solubilized or suspended in an aqueous medium, an organic solvent, a supercritical fluid, or a mixture thereof. Such solution or dispersion is then passed through a certain opening to achieve the desired shape, size, and other properties. Similarly, appropriate drying processes may be used to control the level of the residual dispersing medium, if necessary. The processes, the combination of the processes and/or the modification of the processes described above are well known in the art. Some of the processes are briefly described herein for reference.

Balling. In a broad sense, pellets are very much like granules and bead; the techniques for producing pellets may also produce granules, beads, etc. Pellets, granules or beads are formed with the aid of, e.g., a pelletizer, a spheronizer or an extruder. The pelletizer, spheronizer or extruder is able to form approximately spherical bodies from a mass of finely divided particles continuously, by a rolling or tumbling action on a flat or curved surface with the addition of a liquid.

Pelletizers are generally classified based on the angle of their axis as a horizontal drum or an inclined dish pelletizer. Rotary fluidized granulators may also be used for pelletization. A standard fluidized drier bowl may be replaced with a rotating plate as an air distributor. For granulation, a binder liquid is sprayed from via one or two binary nozzles located axially to the rotational movement of the powder bed. The granulation results in rounding of the granules to approximately spherical pellets. Such balling or agitation techniques are generally influenced by operating conditions, e.g., the bridging/binding liquid requirements, the residence time of the material in the pelletizer, the speed and angle of inclination of the pelletizer, the amount of material fed to the pelletizer and the choice and levels of binder, etc. Those skilled in the art may adjust readily such factors to produce a satisfactory product.

The choice of binder for a given application may also be determined readily by those skilled in the art. Generally, the binder must be capable of wetting the surfaces of the particle being pelletized or granulated. In general, binders must have sufficient wet strength to allow agglomerates to be handled and sufficient dry strength to make them suitable for their intended purposes. Each process, however, makes use of a different system of forces and may require a different agglomerate strength. The final selection of the binder is made generally based on the type of equipment used. Factors that affect the equipment and binder choices include: the size and size distribution of pellets, bulk density, strength and flow properties. Other factors that affect the performance of the pellets, which may be adjusted by one skilled in the art by the inclusion of additives, choice of equipment and processing conditions.

### EXAMPLES

Example 1

The amount of active dissolution over time, e.g., guaifenesin and/or phenylephrine in the tablets or capsules disclosed herein below may be tested as follows. Briefly, in vitro guaifenesin or phenylephrine release may be determined using an acid/base dissolution bath, e.g., a standard USP 23/NF Drug Release Apparatus. Dissolution vessels of a USP calibrated dissolution bath, equipped with shafts and paddles, are filled with 675 ml of 0.1N hydrochloric acid at 37.0 degrees Centigrade. The bath and vessels are maintained at a temperature of 37.0±0.5 degrees Centigrade throughout a standard 7 hour dissolution test. The paddles were set to rotate at 50 RPM and slowly lowered into the vessels. One tablet or capsule is dropped into each vessel.

At the testing intervals, e.g., 1 minute, 10, 10, 30, 45, 60 or 90 minutes, 2, 3, 4, 6, 7, 9 or 12 hour testing intervals, an aliquot, e.g., 5 mls of dissolution solution is withdrawn from each vessel, filtered (e.g., through a 10-22 micron polyethylene filter) and tested using an HPLC. To stop the dissolution a strong base may be added to the sample, e.g., 0.2M sodium phosphate tribasic to increase the pH of the solution to about 6.8. The percent dissolution is determined using HPLC.

Capsule shells and process: 7.5% phenylephrine immediate release beads where used as starting material. A portion of this lot was transferred to a rotating pan. Phenylephrine was added to the beads using of pharmaceutical glaze. The beads were then allowed to roll and cure for 6 hours before sustained release coating was added. In-order to develop the product, four different levels of sustained release coating amounts were added. In one example, 10.93 Kgs of phenylephrine were added to the beads using 4.32 Kgs of pharmaceutical glaze. The beads were then allowed to roll and cure for 6 hours before sustained release coating was added.

In order to develop the product four different levels of sustained release coating amounts were added. The first was 7.15 kg's of SR mix #1 and 4.96 kg's of pharmaceutical glaze. Once this loading was complete 5.0 kg's were removed for drying and testing. The second load consisted of 4.75 kg's of SR mix #1 and 2.68 kg's of pharmaceutical glaze. Again 5.0 kg's of beaded material was removed for drying at 40° C. and testing. The third load consisted of 5.92 kg's SR mix #1 and 3.43 kg's of glaze. After application another 5.0 kg's of beaded material was removed from the pan for drying at 40° C. and testing. The fourth and final load consisted of 7.78 kg's of SR mix #1 and 4.56 kg's of pharmaceutical glaze. The entire pan was allowed to roll and cure under heat lamps for 6 hours before sampling for study.

Table 1 is a list of all theoretical percentages and actual assay results for the, above, described material.

| SR Mix | Theoretical PEH % | Actual PEH % | Diss. 90 min, 3 hr, 6 hr |
|---|---|---|---|
| #1 | 21.6% | 20.8% | 4.6%, 18.6%, 59.3% |
| #2 | 19.8% | 19.3% | 0.2%, 0.8%, 11.0% |
| #3 | 17.8% | 17.3% | 0.16%, 0.4%, 2.7% |
| #4 | 15.5% | 15.4% | 0.6%, 0.8%, 2.6% |

Based on assay and dissolution profile load #1 was selected for use in further development. The moisture content in load #4 may be higher than those loads dried in the tray drier. This may have contributed to why load #3 and #4 have essentially the same dissolution profile despite the increased SR mix. The gauifenesin DC 95% was compressed into slugs using a bb2 type tablet press with standard ¼″ cup tooling. GRA001 was pressed into slugs weighing 220 mg each. Capsules were filled using 75 mg of Load #1 beads (15 mg phenylephrine). Then two 220 mg slugs of Gauifenesin DC. These capsule were then placed in a 75 cc bottle and conditioned at 35° C. for 24 hours. Dessicant was then added. The material was capped and the induction seal was activated. The material was placed on accelerated stability.

Dissolution: The present inventors found that the dissolution rate of the phenylephrine is accelerated when combined with Gauifenesin DC. Due to this effect the testing of the dissolution rate is achieved by first making a mock-up of the finished product. By doing so the suitability of the phenylephrine beads was determined more accurately. Direct specifications for dissolution were determined once data was collected to accurately predict this rate change.

Stability: Capsules were studied for stability. Accelerated stability indicates that the product is stable. Gauifenesin DC released 100% immediately with stable potency. The phenylephrine exhibited a first order release profile consistent with an 8 hour product and was consistent from month to month. Results are summarized below in Table 2.

| Lot # | Count | Container | Closure | Desiccant |
|---|---|---|---|---|
| DEV 191 | 100 | CON014 | CLO452 | DES101 |

| | GFN | PEH Diss. | PEH |
|---|---|---|---|
| Time | Diss. 90 min/Assay | 90 min/3 hr/6 hr | Assay |
| Initial | 103.7%/99.8% | 30.1%/43.5%/70.5% | 105.2% |
| 4 week | 103.5%/98.5% | 33.8%/49.4%/78.7% | 105.7% |
| 6 week | 104.2%/98.7% | 20.8%/38.3%/70.6% | 100.4% |
| 8 week | 102.6%/99.5% | 26.8%/44.5%/78.7% | 107.3% |
| 10 week | 103.9%/99.4% | 31.5%/46.2%/73.1% | 97.8% |
| 12 week | 104.1%/98.8% | 33.7%/51.0%/85.5% | 107.7% |

Equipment: Bosch GKF 700 and GKF 2000 machines were used for the pellet and powder functions. The GKF 700 runs the 400/15 product that requires the beads to be dosed prior to powder. The GKF 2000 runs product 200/7.5 and is capable of filling powder followed by beads. The reason for this is that the 400/15 product is in a size 0 elongated capsule that is overfilled. If the beads are added after the powder slug they will tend to roll off the slug during capsule closure. This would result in poor closure and poor content uniformity.

### Example 2

Phenylephrine for delayed release may be prepared using pharmaceutical glaze, polyvinylpyrrolidone and/or microcrystalline cellulose in combination with one or more inactive agents. For example, the phenylephrine may be allowed to roll and cure for 1-6 hours in the presence of the polyvinylpyrolidone and microcrystalline cellulose. Optionally, a sustained release coating may be added to infuse and/or coat the active-polymer (phenylephrine-polyvinylpyrrolidone). Different levels of sustained release coating amounts may be added, with or without intervening layers of active and/or polymer. In one example, 10.93 Kgs of phenylephrine may be added to polyvinylpyrrolidone and pharmaceutical glaze. The phenylephrine-polyvinylpyrrolidone is allowed to roll and cure for 1-6 hours before sustained release coating (pharmaceutical glaze) is added.

Table 3 is a list of all percentages of actual assay results for the above described formulation for extended release phenylephrine.

| PHFB | | | | |
|---|---|---|---|---|
| DVR II | Phenylephrine | 90 min | 3 hours | 6 hours |
| ELA | 30 mg | 41.5 | 58.0 | 80.3 |
| GFD | 30 mg | 57.6 | 69.9 | 86.4 |
| EER | 30 mg | 36.2 | 51.2 | 74.6 |

Table 4 shows the release profile for guaifenesin immediate release.

| PHFB | | | | |
|---|---|---|---|---|
| DVR II | Guaifenesin | 90 min | 3 hours | 6 hours |
| ELA | 400 mg | 100.9 | N/A | N/A |
| GFD | 400 mg | 99.7 | N/A | N/A |
| EER | 400 mg | 101.4 | N/A | N/A |

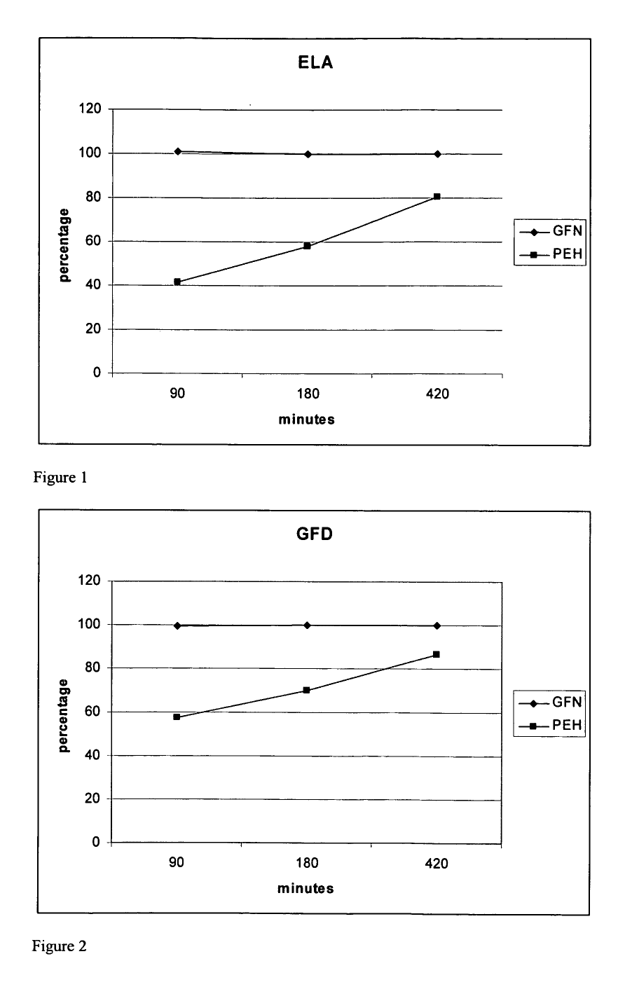

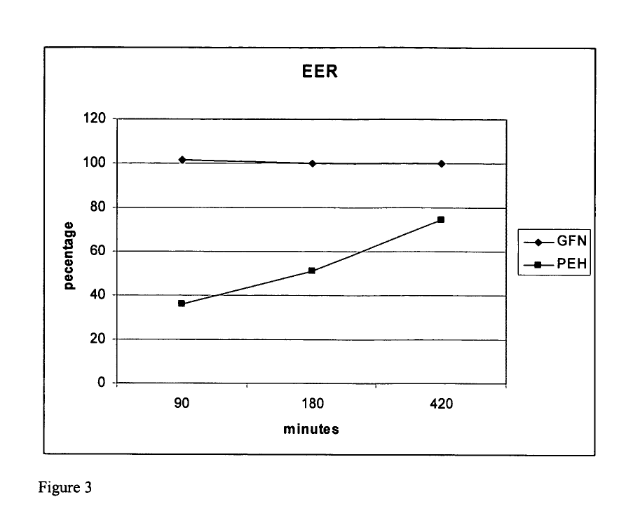

The results from the formulations are summarized in FIGS. 1-3, which demonstrate the percentage release of the three different formulations and packaging of actives over time. The gauifenesin DC 95% may be compressed into slugs using, e.g., a bb2 type tablet press with standard ¼″ cup tooling. GRA001 was pressed into slugs weighing 220 mg each. Capsules may be filled with phenylephrine and two 220 mg slugs of Gauifenesin DC. These capsules are placed in a 75 cc bottle and conditioned at 35° C. for 24 hours. Dessicant was then added. The material was capped and the induction seal was activated. The material was placed on accelerated stability.

Dissolution: The present inventors found that the dissolution rate of the phenylephrine is accelerated when combined with Gauifenesin DC. Due to this effect the testing of the dissolution rate is achieved by first making a mock-up of the finished product. By doing so the suitability of the phenylephrine was determined more accurately. Direct specifications for dissolution were determined once data was collected to accurately predict this rate change.

Stability: Capsules were studied for stability. Accelerated stability indicates that the product is stable. Gauifenesin DC released 100% immediately with stable potency. The phenylephrine exhibited a first order release profile consistent with an 8 hour product and was consistent from month to month. Results are summarized below.

Formula I. A batch of immediate release expectorant, e.g., guaifenesin for use with the enveloped formulation was prepared with the following components:

| Components | Weight |
|---|---|
| Guaifenesin DC | 421 mg |
| Talc | 5 mg |

Formula II. A batch of immediate release guaifenesin for use with the enveloped formulation was prepared with the following components:

| Components | Weight |
|---|---|
| Guaifenesin DC | 632 mg |
| Talc | 3 mg |
| Stearic Acid | 2 mg |

Formula III. A batch of immediate release guaifenesin for use with the enveloped formulation was prepared with the following components:

| Components | Weight |
|---|---|
| Guaifenesin DC | 211 mg |
| Talc | 3 mg |
| Magnesium Stearate | 2 mg |

Formula IV. A batch of immediate release guaifenesin for use with the enveloped formulation was prepared with the following components:

| Components | Weight |
|---|---|
| Guaifenesin DC | 421 mg |
| Magnesium Stearate | 3 mg |
| Ludipress | 50 mg |

Formula IV. A batch of effervescent expectorant for immediate release, e.g., guaifenesin for use with the enveloped formulation was prepared with the following components:

| Components | Weight |
|---|---|
| Guaifenesin DC | 421 mg |
| Talc | 5 mg |
| Sodium bicarbonate | 25 mg |

When combining the first and the second active, these may be formulated as follows. A capsule for immediate release of a first active and extended release of a second active in an enveloped formulation, in a single capsule:

| First Active | Weight | | Second Active | Weight | |
|---|---|---|---|---|---|
| Guaifenesin DC | 421 | mg | Phenylephrine | 15 | mg |
| Talc | 5 | mg | Bead | 44 | mg |
| | | | Lacquer | 6 | mg |
| | | | Talc | 5 | mg |
| | | | Calcium Stearate | 5 | mg |
| Capsule | 1 | | | | |

A formulation for immediate release of a first active and extended release of a second active in an enveloped formulation, in a gelcap:

| First Active | Weight | Second Active | Weight |
|---|---|---|---|
| Guaifenesin DC | 421 mg | Phenylephrine | 15 mg |
| Talc | 0 mg | Bead | 44 mg |
| | | Lacquer | 6 mg |
| | | Talc | 5 mg |
| | | Calcium Stearate | 5 mg |
| Gelcap | 1 | | |

A formulation for immediate release of a first active and extended release of a second active in an enveloped formulation, in a suppository:

| First Active | Weight | | Second Active | Weight |
|---|---|---|---|---|
| Guaifenesin DC | 421 | mg | Phenylephrine | 5 mg |
| Talc | 5 | mg | Bead | 15 mg |
| | | | Lacquer | 2 mg |
| | | | Talc | 1.5 mg |
| | | | Calcium Stearate | 1.5 mg |
| Stearic Acid | 2 | mg | | |
| beeswax/glycerol | 1-2 | gr | | |

An effervescent tablet for immediate release of a first active and extended release of a second active in an enveloped formulation, in an effervescent tablet:

| First Effervescent Active | Weight | Second Active Minicap | Weight |
|---|---|---|---|
| Guaifenesin DC | 421 mg | Phenylephrine | 15 mg |
| Talc | 5 mg | Bead | 44 mg |
| | | Lacquer | 6 mg |
| | | Talc | 5 mg |
| | | Calcium Stearate | 5 mg |
| Monosodium citrate | 10 mg | | |
| Sodium bicarbonate | 10 mg | | |

For immediate release of a first active and extended release of a second active in an enveloped formulation one may add the following ingredients, in a caplet:

| First Active | Weight | Second Active | Weight |
|---|---|---|---|
| Guaifenesin DC | 421 mg | Phenylephrine | 15 mg |
| Talc | 3 mg | Bead | 44 mg |
| | | Lacquer | 6 mg |
| | | Talc | 5 mg |
| | | Calcium Stearate | 5 mg |

When combining the first active and the decongestant, these may be formulated as follows. A capsule for immediate release of an expectorant and extended release of a decongestant in an enveloped formulation, in a single capsule:

| Expectorant | Weight | Decongestant | Weight |
|---|---|---|---|
| Guaifenesin DC | 421 mg | Phenylephrine HCl | 30 mg |
| Talc | 5 mg | PVPyrrole | 44 mg |
| | | Povidone | 6 mg |
| | | Talc | 5 mg |
| | | Calcium Stearate | 5 mg |
| Capsule | 1 | | |

When combining the first active and the decongestant, these may be formulated as follows. A capsule for immediate release of an expectorant and extended release of a decongestant in an enveloped formulation, in a single capsule:

| Expectorant | Weight | Decongestant | Weight |
|---|---|---|---|
| Guaifenesin DC | 421 mg | Phenylephrine HCl | 30 mg |
| Talc | 5 mg | PVPyrrole | 44 mg |
| | | Povidone | 6 mg |
| | | Microcrystalline Cellulose | 5 mg |
| | | Magnesium Stearate | 5 mg |
| Gelatin Capsule | 1 | | |

A formulation for immediate release of an expectorant and extended release of a decongestant in an enveloped formulation, in a gelcap:

| Expectorant | Weight | Decongestant | Weight |
|---|---|---|---|
| Guaifenesin DC | 421 mg | Phenylephrine | 30 mg |
| | | Microcrystalline Cellulose | 44 mg |
| | | Magnesium Stearate | 5 mg |
| Gelcap | 1 | | |

A formulation for immediate release of an expectorant and extended release of a decongestant in an enveloped formulation, in a suppository:

| Expectorant | Weight | | Decongestant | Weight |
|---|---|---|---|---|
| Guaifenesin DC | 421 | mg | Phenylephrine | 15 mg |
| Talc | 5 | mg | PVPyrrole | 44 mg |
| | | | Povidone | 6 mg |
| | | | Microcrystalline Cellulose | 5 mg |
| | | | Magnesium Stearate | 5 mg |
| Stearic Acid | 2 | mg | | |
| beeswax/glycerol | 1-2 | gr | | |

An effervescent tablet for immediate release of an expectorant and extended release of a decongestant in an enveloped formulation, in an effervescent tablet:

| First Effervescent Active | Weight | Decongestant Minicap | Weight |
|---|---|---|---|
| Guaifenesin DC | 421 mg | Phenylephrine | 15 mg |
| Talc | 5 mg | PVPyrrole | 44 mg |
| | | Povidone | 6 mg |
| | | Microcrystalline Cellulose | 5 mg |
| | | Magnesium Stearate | 5 mg |
| Monosodium citrate | 10 mg | | |
| Sodium bicarbonate | 10 mg | | |

For immediate release of an expectorant and extended release of a decongestant in an enveloped formulation one may add the following ingredients, in a caplet:

| Expectorant | Weight | Decongestant | Weight |
|---|---|---|---|
| Guaifenesin DC | 421 mg | Phenylephrine | 15 mg |
| Talc | 3 mg | PVPyrrole | 44 mg |
| | | Povidone | 6 mg |
| | | Microcrystalline Cellulose | 5 mg |
| | | Magnesium Stearate | 5 mg |

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

1. A pharmaceutical composition comprising: an expectorant packaged for release of over 90% within about 90 minutes; and a decongestant packaged for extended release wherein between about 30 to 60% of the decongestant is available after 90 minutes, between about 50 to 70 percent is available at between 150 and 210 minutes and wherein between about 60 to 80 percent is available after 360 minutes.

2. The composition of claim 1, wherein the decongestant comprises phenylephrine.

3. The composition of claim 1, wherein the expectorant comprises guaifenesin.

4. The composition of claim 1, wherein the pharmaceutical formulation is packed into a capsule.

5. The composition of claim 1, wherein the decongestant and the expectorant are packed into a capsule, caplet, softgel, gelcap, suppository, film, granule, gum, insert, pastille, pellet, troche, lozenge, disk, poultice or wafer.

6. The composition of claim 1, wherein over 80% of the expectorant is released within about 60 minutes.

7. The composition of claim 1, wherein immediate release is defined further as comprising release of over 90% of the expectorant within about 60 minutes.

8. The composition of claim 1, wherein the decongestant is packaged with PVPP and Povidone.

9. The con)position of claim 1, wherein the decongestant is packaged with a talc and a stearate.

10. The composition of claim 1, wherein the expectorant comprises gauifenesin in a powder form.

11. The composition of claim 1, wherein the expectorant comprises 422 mg of 95% gauifenesin.

12. The composition of claim 1, wherein the decongestant comprises phenylephrine as a sustained release bead, a layered sustained release bead or three or more layers of phenylephrine on a bead.

13. The composition of claim 1, wherein the expectorant is superposed on the decongestant.

14. The composition of claim 1, comprising further one or more inactives.

15. A pharmaceutical composition consisting essentially of: an expectorant packaged for release of over 90% within about 90 minutes; a decongestant packaged for extended release wherein between about 30 to 60% of the decongestant is available after 90 minutes, between about 50 to 70 percent is available at between 150 and 210 minutes and wherein between about 60 to 80 percent is available after 360 minutes; and one or more inactive agents.

16. A capsule consisting essentially of: an expectorant packaged for release of over 90% within about 90 minutes; a decongestant packaged for extended release wherein between about 30 to 60% of the decongestant is available after 90 minutes, between about 50 to 70 percent is available at between 150 and 210 minutes and wherein between about 60 to 80 percent is available after 360 minutes; and one or more inactive agents.

17. A method of providing a dual-release formulation comprising: loading into a capsule an expectorant in a powered form and a decongestant in an extended release form, wherein the capsule comprises one or more excipients selected from a polymer, a cellulose, a stearate, a talc, a lacquer and a pharmaceutical glaze.

18. A method of providing a dual-release formulation comprising: providing a expectorant packed for immediate release in a powder form; providing a nasal decongestant packed for extended release comprising, phenylephrine, PVP, cellulose and a pharmaceutical glaze, and loading the expectorant and the decongestant into a capsule

19. A pharmaceutical composition consisting essentially of: a guaifenesin packaged for release of over 90% within about 90 minutes; a phenylephrine packed for extended release; and one or more inactive agents selected from a polyvinyl polymer, microcrystalline cellulose, stearate, lacquer, talc and a pharmaceutical glaze, wherein between about 30 to 60% of the decongestant is available after 90 minutes, between about 50 to 70 percent is available at between 150 and 210 minutes and wherein between about 60 to 80 percent is available after 360 minutes.

20. A pharmaceutical composition consisting essentially of: an expectorant packaged for release of over 90% within about 90 minutes; a decongestant packaged for extended release wherein between about 30 to 60% of the decongestant is available after 90 minutes, between about 50 to 70 percent is available at between 150 and 210 minutes and wherein between about 60 to 80 percent is available after 360 minutes; and one or more inactive agents.

21. The composition of claim 20, wherein the decongestant comprises phenylephrine.

22. The composition of claim 20, wherein the expectorant comprises guaifenesin.

23. The composition of claim 20, wherein the pharmaceutical formulation is packed into a capsule.

24. The composition of claim 20, wherein the decongestant and the expectorant are packed into a capsule, caplet, softgel, gelcap, suppository, film, granule, gum, insert, pastille, pellet, troche, lozenge, disk, poultice or wafer.

25. The composition of claim 20, wherein over 80% of the expectorant is released within about 60 minutes.

26. The composition of claim 20, wherein immediate release is defined further as comprising release of over 90% of the expectorant within about 60 minutes.

27. The composition of claim 20, wherein the decongestant is packaged with a polyvinyl polymer and Povidone.

28. The composition of claim 20, wherein the decongestant is packaged with a talc and a stearate.

29. A pharmaceutical composition comprising: a guaifenesin salt packaged for release of over 90% within about 90 minutes; a phenylephrine salt packaged for extended release wherein between about 30 to 60% of the decongestant is available after 90 minutes, between about 50 to 70 percent is available at between about 150 and 210 minutes and wherein between about 60 to 80 percent is available after 360 minutes.

30. The composition of claim 29, wherein the decongestant comprises phenylephrine.

31. The composition of claim 29, wherein the expectorant comprises guaifenesin.

32. The composition of claim 29, wherein the pharmaceutical formulation is packed into a capsule.

33. The composition of claim 29, wherein the decongestant and the expectorant are packed into a caplet, softgel, gelcap, suppository, film, granule, gum, insert, pastille, pellet, troche, lozenge, disk, poultice or wafer.

34. The composition of claim 29, wherein over 80% of the expectorant is released within about 60 minutes.

35. The composition of claim 29, wherein immediate release is defined further as comprising release of over 90% of the expectorant within about 60 minutes.

36. The composition of claim 29, wherein the decongestant is packaged with a polyvinyl polymer and Povidone.

37. The composition of claim 29, wherein the decongestant is packaged with a talc and a stearate.
